(19) United States
(12) Patent Application Publication
(10) Pub. No.: US 2025/0152397 A1
(43) Pub. Date: May 15, 2025

(54) OSTOMY POUCH DEVICE AND METHOD

(71) Applicant: BOSTON SCIENTIFIC SCIMED, INC., Maple Grove, MN (US)

(72) Inventors: Rosangel Aimee RAMOS ESPINOZA, Boston, MA (US); Serena SCOTT, Worcester, MA (US); Estefan MORA SOJO, Chicago, IL (US); Brian LUIS, Boylston, MA (US); Pauline Rosemary LIMBERG, Northborough, MA (US); Juan M. ALVAREZ AZPEITIA, Framingham, MA (US)

(21) Appl. No.: 18/923,051

(22) Filed: Oct. 22, 2024

Related U.S. Application Data
(60) Provisional application No. 63/599,422, filed on Nov. 15, 2023.

(51) Int. Cl. A61F 5/44; A61F 5/445

(52) U.S. Cl. CPC A61F 5/4404; A61F 5/445

(57) ABSTRACT

An ostomy device includes a pouch, a bag, and a securing mechanism. The pouch extends from a proximal end attachable to skin surrounding a stoma exit of a patient to a distal end. The pouch includes an outlet accessible to the patient. The bag is housed within the pouch and extends from a proximal end interfacing with the exit to a distal end. The bag includes separatable sections. The mechanism secures, relative to the pouch, remaining sections that are proximal of a distal-most section. The bag is configured so that, when the distal-most section is filled with waste from the exit, the distal-most section may be detached from the remaining sections and a distal-most one of the remaining sections is securable by the mechanism relative to the pouch in a position to interface with the exit to receive further waste from the exit.

### PRIORITY CLAIM

The present disclosure claims priority to U.S. Provisional Patent Application Ser. No. 63/599,422 filed Nov. 15, 2023; the disclosure of which is incorporated herewith by reference.

### FIELD

The present disclosure relates to ostomy pouch devices and methods for post-procedural ostomy management.

### BACKGROUND

An ostomy refers to a surgical procedure in which an opening, or stoma, is created in the abdomen to provide a path for human waste or a stool (e.g., fluid, solid and/or gaseous waste) to be removed from the body. Sections of the gastrointestinal tract are bypassed so that the waste typically transported through the body to a natural orifice (e.g., the anus) is excreted through the stoma. Traditionally, ostomies are managed through technology such as an ostomy bag or pouch attached to the stoma to catch the human waste or stool drained therefrom. The patient must regularly vacate and replace the ostomy bag as it becomes full.

Most ostomy bags need to be emptied and cleaned or replaced once they are full of effluent (e.g., human waste and/or stool). The patient must empty and clean or replace the ostomy bag four to ten times per day. This includes disconnecting the bag from the skin or skin barrier, opening the bag, using the outlet to empty the effluent, cleaning the bag, closing the outlet, and re-attaching the bag. This process can be difficult, tiring, and unpleasant for ostomy patients as they get rid of the effluent.

### SUMMARY

The present disclosure relates to an ostomy pouch device. The device includes an ostomy pouch extending from a proximal end configured to attach to skin surrounding a stoma exit of a patient to a distal end, the ostomy pouch including an outlet accessible to the patient; an ostomy bag housed within the ostomy pouch, the bag extending from a proximal end configured to interface with the stoma exit to a distal end, the bag including a plurality of sections separatable from one another; and a securing mechanism configured to secure, relative to the ostomy pouch, remaining sections of the bag that are proximal of a distal-most section of the bag. The bag is configured so that, when the distal-most section is filled with waste from the stoma exit, the distal-most section may be detached from the remaining sections and a distal-most one of the remaining sections is configured to be secured by the securing mechanism relative to the ostomy pouch in a position to interface with the stoma exit to receive further waste from the stoma exit.

In an embodiment, the securing mechanism includes a cap configured to couple the distal end of the bag to the distal end of the ostomy pouch.

In an embodiment, the cap includes an adhesive to permanently attach the cap to the distal end of the bag.

In an embodiment, at least one of the cap and the distal end of the ostomy pouch includes a coupling mechanism configured to couple the cap to the distal end of the bag.

In an embodiment, the outlet is an opening in the distal end of the ostomy pouch.

In an embodiment, the securing mechanism includes an iris diaphragm configured to be actuated between an open configuration, in which blades of the iris diaphragm are rescinded therein, and a closed configuration, in which the blades of the iris diaphragm are extended radially therefrom.

In an embodiment, when the iris diaphragm is in the closed configuration, the blades tighten a proximal end of the distal-most section to isolate the distal-most section from the remaining sections.

In an embodiment, the iris diaphragm includes at one of a heating element and a cutting element so that, when the iris diaphragm is in the closed configuration, the blades tighten a proximal end of the distal-most section to at least one of seal the distal-most section from the remaining sections and detach the distal-most section from the remaining sections.

In an embodiment, the securing mechanism includes a plurality of flanges configured to be actuated between a tightened configuration, in which flanges secure the remaining sections against a stoma base plate of the ostomy pouch, and a loosened configuration, in which the flanges release at least a portion of the remaining sections.

In an embodiment, the device further includes a closing device configured to seal a proximal end of the distal-most section.

In an embodiment, the device further includes a plurality of further closing devices each configured to seal at least one of a distal end of the distal-most section, a proximal end of each one of the remaining sections, and a distal end of each one of the remaining sections.

In an embodiment, the device further includes an irrigation hole configured to be penetrated by an irrigation device to irrigate the stoma exit and a surrounding area of the stoma exit.

In an embodiment, the irrigation hole is distal of a proximal end of the distal-most section.

In addition, the present disclosure relates to an ostomy pouch device. The device an ostomy pouch including a base plate configured to attach to skin surrounding a stoma exit, the ostomy pouch including an outlet; an ostomy bag housed within the ostomy pouch and configured to interface with the stoma exit, the bag including a plurality of sections, each of the sections movable from a compacted configuration, in which the section is unable to receive waste therein, to an expanded configuration, in which the section is able to receive waste therein; and a securing mechanism configured to secure at least one of a first one of the sections and a second one of the sections, relative to the ostomy pouch, the first one of the sections being distal of the second one of the sections. The bag is configured so that, when the first one of the sections is filled with waste from the stoma exit, the first one of the sections may be detached from the second one of the sections, and the second one of the sections is secured by the securing mechanism relative to the ostomy pouch and is moved from the compacted configuration to the expanded configuration to receive further waste from the stoma exit.

In addition, the present disclosure relates to a method. The method includes attaching to a stoma exit an ostomy pouch of an ostomy pouch device, the ostomy pouch device comprising an ostomy bag housed inside the ostomy pouch including a plurality of sections separatable from one another, each of the sections movable from a compacted configuration, in which the sections are unable to receive waste therein, to an expanded configuration, in which the sections are able to receive waste therein and a securing mechanism configured to secure, relative to the ostomy pouch, remaining sections of the bag that are proximal of a distal-most section of the bag; opening an outlet of the ostomy pouch to access the bag and the securing mechanism; actuating the securing mechanism to isolate the distal-most section; sealing a proximal end of the distal-most section from the remaining sections; detaching the distal-most section from the remaining sections; securing, by the securing mechanism, relative to the ostomy pouch a first one of the remaining sections; moving the first remaining section into the expanded configuration to receive the waste therein; and sealing the outlet of the ostomy pouch.

In an embodiment, the step of sealing the proximal end of the distal-most section comprises attached a closing device onto the proximal end of the distal-most section.

In an embodiment, the method further includes attaching a closing device to a distal end of the first one of the remaining sections when the first one of the remaining sections is moved into the expanded configuration.

In an embodiment, the method further includes inserting an irrigation device into an irrigation hole in the bag to irrigate the stoma exit and a surrounding area of the stoma exit.

In an embodiment, the securing mechanism includes one of (a) a cap configured to couple a distal end of the bag to a distal end of the ostomy pouch, (b) an iris diaphragm configured to be actuated between an open configuration in which blades of the iris diaphragm are rescinded therein and a closed configuration in which the blades of the iris diaphragm are extended radially therefrom to tighten the proximal end of the distal-most section and isolate the distal-most section from the remaining sections, and (c) a plurality of flanges configured to be actuated between a tightened configuration in which flanges secure the remaining sections against a stoma base plate of the ostomy pouch, and a loosened configuration in which the flanges release at least a portion of the remaining sections.

In an embodiment, the distal-most section is detached from the remaining sections by cutting or tearing along the proximal end of the distal-most section.

### BRIEF DESCRIPTION

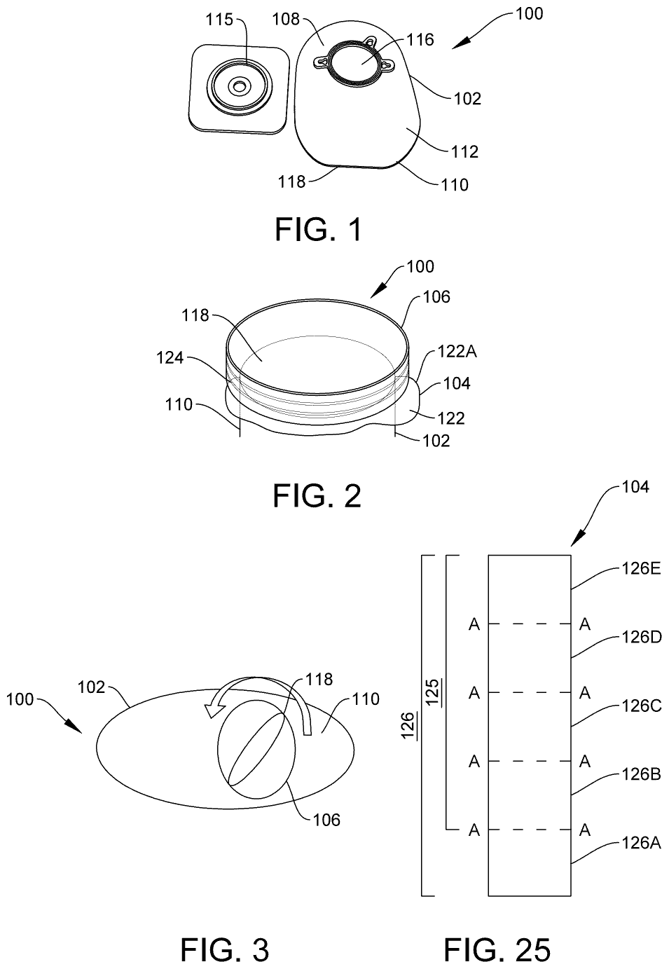

FIG. 1 shows a perspective view of an ostomy pouch device according to a first exemplary embodiment of the present disclosure.

FIG. 2 shows a side view of a cap of the ostomy pouch device of FIG. 1 coupled onto an ostomy bag and an ostomy pouch of the ostomy pouch device of FIG. 1.

FIG. 3 shows a back view of the ostomy pouch device of FIG. 1.

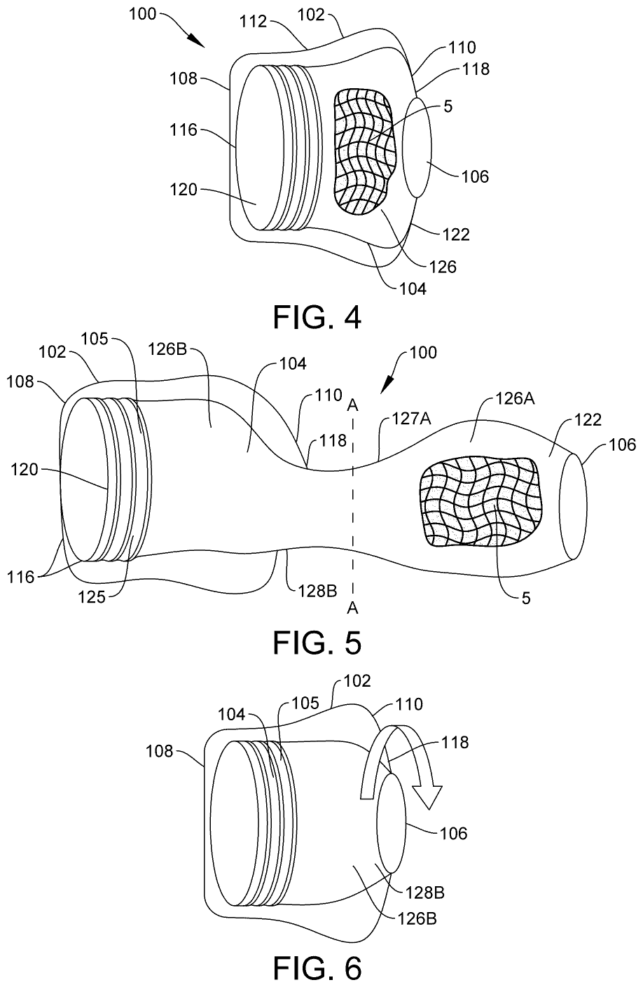

FIG. 4 shows a semi-transparent side view of an empty distal-most section of an ostomy bag within an ostomy pouch of the ostomy pouch device of FIG. 1.

FIG. 5 shows a semi-transparent side view of a filled distal-most section of the ostomy bag pulled out of the ostomy pouch of the ostomy pouch device of FIG. 1.

FIG. 6 shows a semi-transparent side view of an empty new distal-most section of the ostomy bag within the ostomy pouch of the ostomy pouch device of FIG. 1.

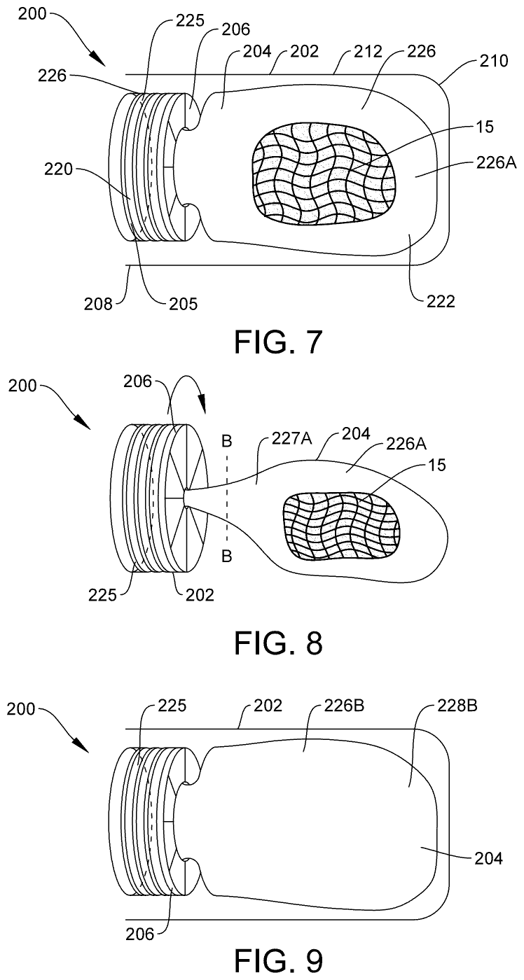

FIG. 7 shows a semi-transparent side view of an ostomy pouch device according to a second exemplary embodiment of the present disclosure.

FIG. 8 shows a semi-transparent side view of a filled distal-most section of the ostomy bag pulled out of the ostomy pouch of the ostomy pouch device of FIG. 7.

FIG. 9 shows a semi-transparent side view of an empty new distal-most section of the ostomy bag within the ostomy pouch of the ostomy pouch device of FIG. 7.

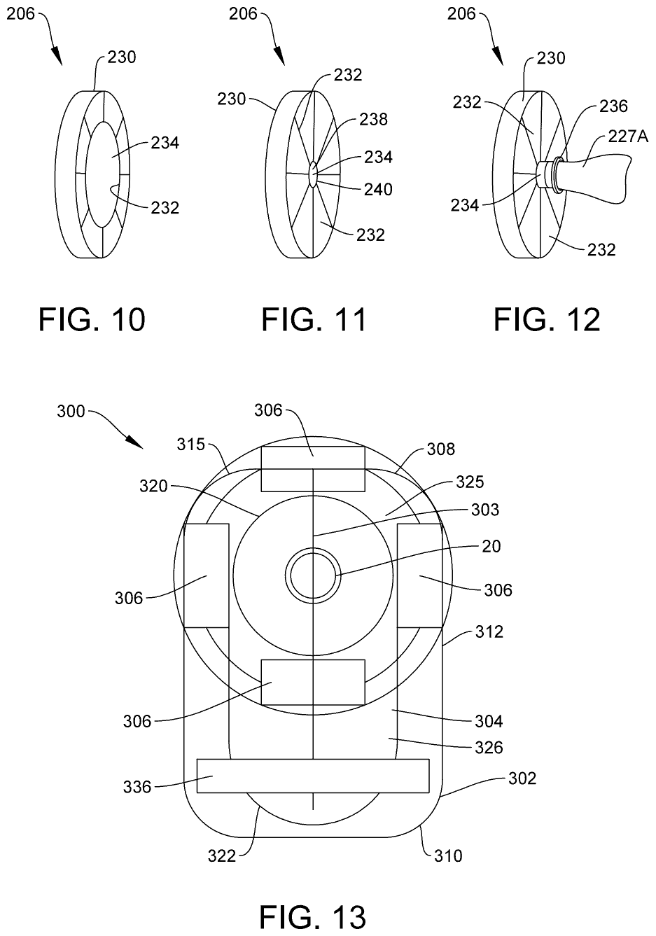

FIG. 10 shows a perspective view of an iris diaphragm of the ostomy pouch device of FIG. 7 in an open configuration.

FIG. 11 shows a perspective view of the iris diaphragm of FIG. 10A in a closed configuration.

FIG. 12 shows a perspective view of a portion of the ostomy bag being tightened by the iris diaphragm of FIG. 10A in the closed configuration.

FIG. 13 shows a front view of an ostomy pouch device according to a third exemplary embodiment of the present disclosure.

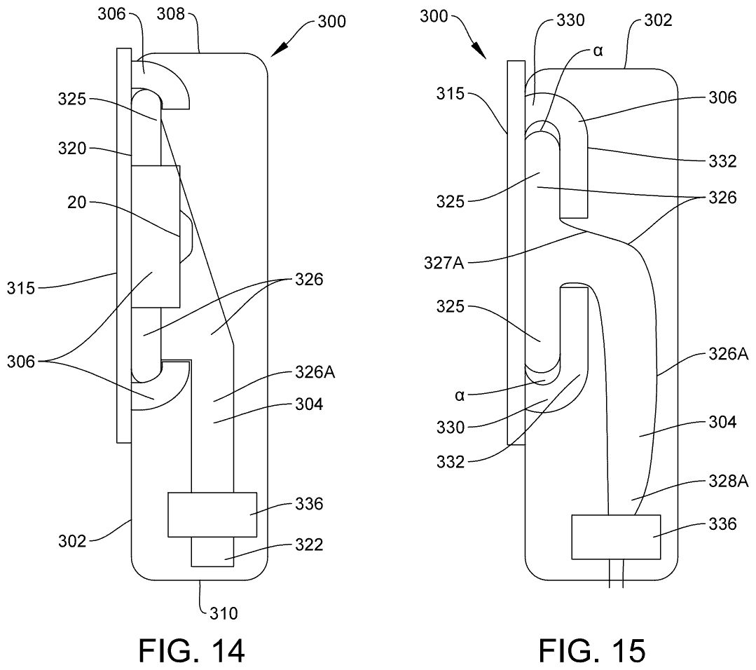

FIG. 14 shows a side view of the ostomy pouch device of FIG. 13.

FIG. 15 shows a side view of a cross section of the ostomy pouch device of FIG. 13.

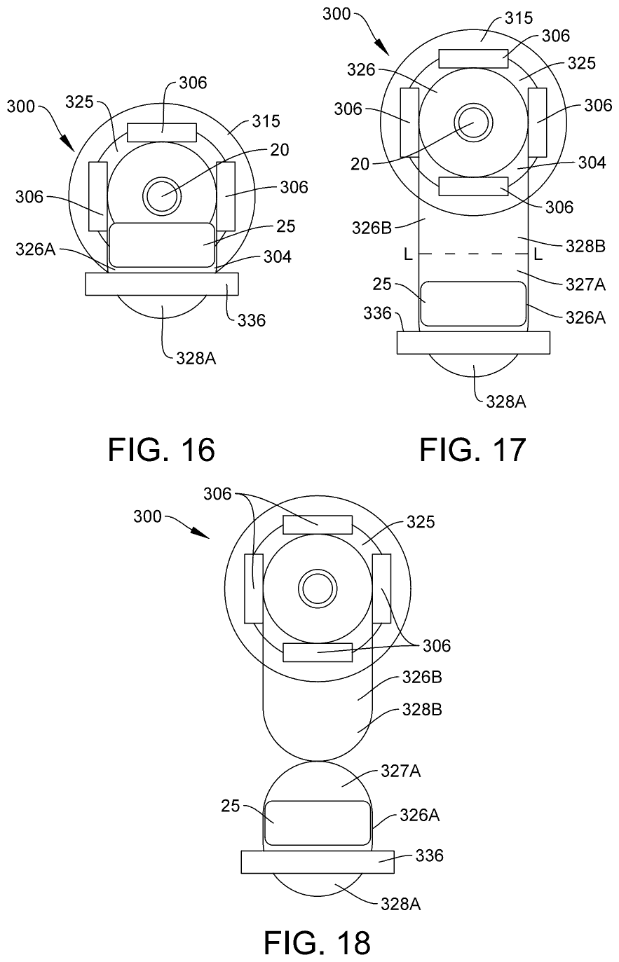

FIG. 16 shows a semi-transparent front view of a filled distal-most section of an ostomy bag of the ostomy pouch device of FIG. 13 with flanges tightened.

FIG. 17 shows a semi-transparent front view of the filled distal-most section of FIG. 16 with the flanges loosened.

FIG. 18 shows a semi-transparent front view of the filled distal-most section of FIG. 16 twisted away from a remaining portion of the ostomy bag.

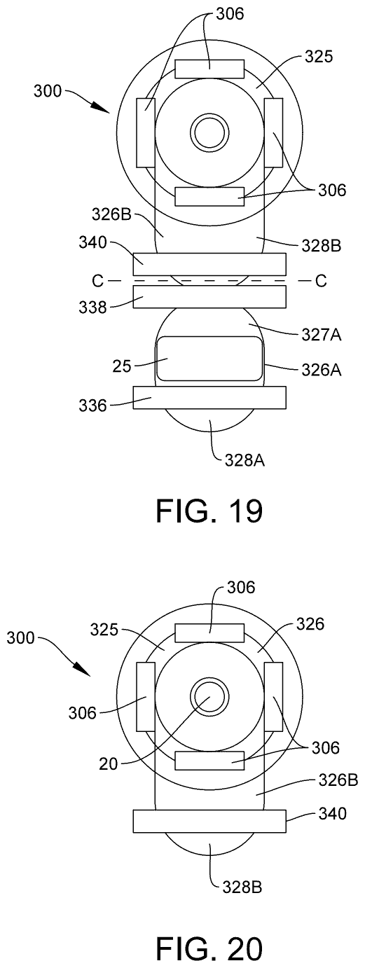

FIG. 19 shows a semi-transparent front view of the filled distal-most section of FIG. 16 clamped and tied.

FIG. 20 shows a semi-transparent front view of the remaining portion of the ostomy bag clamped and separated from the filled distal-most section of FIG. 16.

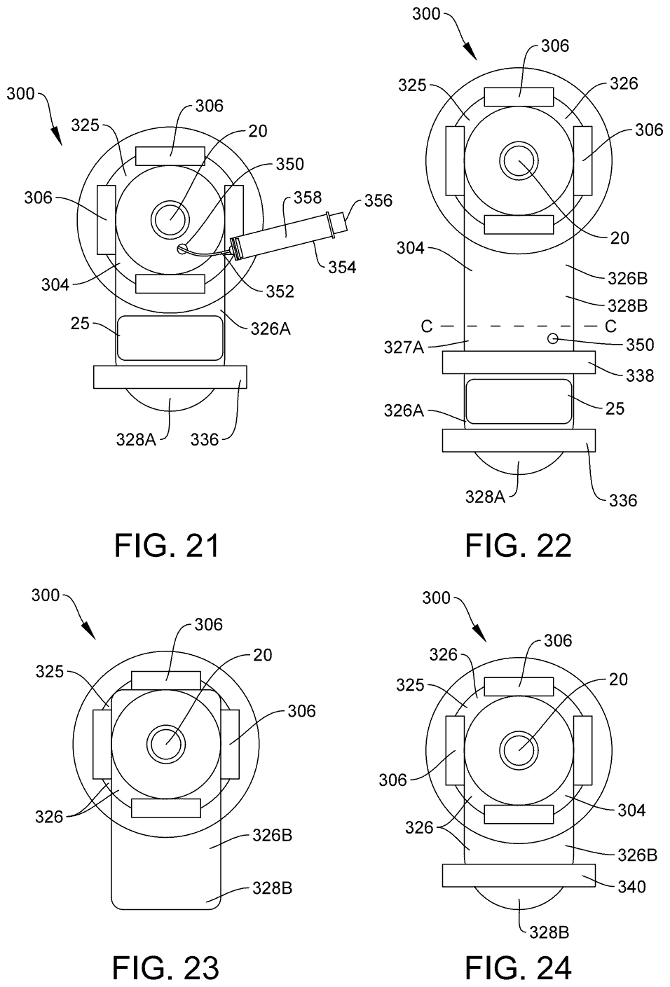

FIG. 21 shows a semi-transparent front view of an ostomy bag of the ostomy pouch device of FIG. 13 having a hole for irrigation by a syringe.

FIG. 22 shows a semi-transparent front view of the ostomy bag of FIG. 21 with flanges loosened.

FIG. 23 shows a semi-transparent front view of a remaining portion of the ostomy bag of FIG. 21 after a filled distal-most section of the ostomy bag was removed.

FIG. 24 shows a semi-transparent front view of the remaining portion of the ostomy bag of FIG. 21 clamped and with the flanges tightened.

FIG. 25 shows a top view of an ostomy bag of the ostomy pouch device of FIG. 1.

### DETAILED DESCRIPTION

The present disclosure may be further understood with reference to the following description and the appended drawings, wherein like elements are referred to with the same reference numerals. Exemplary embodiments of the present disclosure relate to an ostomy device comprising an ostomy pouch, an ostomy bag including multiple sections, and a securing mechanism configured to permit a patient to remove and discard a section of the ostomy bag when it is full without having to clean the ostomy pouch by separating the full section of the ostomy bag from remaining unused sections of the ostomy bag.

The detached full section of the ostomy bag is discarded and a portion of the remaining unused ostomy bag is positioned to receive waste from the stoma. This process is repeated until the entire ostomy bag has been used at which point a new ostomy bag is attached to the pouch adjacent to the stoma to receive the waste therefrom. The ostomy device, thus, reduces odors helping keep the ostomy pouch low profile. In addition, the ostomy pouch may have an outlet that is easy to open, and it may include an inspection window to help the patient determine when the currently filling section of the ostomy bag should be detached and discarded.

There are several types of ostomy procedures including a colostomy, an ileostomy and a urostomy. In a colostomy, portions of the large intestine (e.g., colon and/or rectum) are removed and a portion of the remaining tissue is brought through an opening in the abdomen to form the stoma. In an ileostomy, the ileum (end of the small intestine) is brought through the abdomen to form the stoma. After one of these procedures has been performed, waste is passed through the stoma for collection (e.g., in an ostomy bag). In a urostomy, a portion of the small intestine is detached from the rest of the small intestine and brought through the abdomen to form a stoma. This detached portion of the small intestine is coupled to the ureters to allow for the passage of urine away from the bladder and out of the stoma to an ostomy bag. Some ostomy procedures can be performed laparoscopically using small incisions while others are performed as open surgeries using a larger incision. Some ostomy procedures are permanent while others are reversible. Different procedures can require different durations for which the ostomy bag must be used.

The stoma may be flat against the skin or may protrude a small distance out of the abdomen. The pouching system for receiving the waste via the stoma may, for example, comprise a one-piece ostomy bag attaching directly to the abdomen around the stoma to form a seal or may comprise a two-piece system including a flange attached to the abdomen with the ostomy bag attached to the flange to form the seal. The ostomy bag has an entrance opening on or adjacent to its top side for interfacing with the stoma and receiving the waste. The ostomy bag can be externally supported at the abdomen, e.g., by adhesive or by using a belt to which the ostomy bag attaches on its top side. Ostomy bags come in a variety of different sizes, shapes and styles. For example, some ostomy bags lie flat against the skin so that the bag can be worn discreetly for daily use.

FIGS. 1-6 and 25 show an ostomy pouch device 100 according to a first exemplary embodiment of the present disclosure. The pouch device 100 includes an ostomy pouch 102, an ostomy bag 104 and a cap 106. The ostomy pouch 102 includes a first side 108 (e.g., a top side when the ostomy pouch 102 is oriented in its intended manner on the body of the patient) attachable to the body of the patient, a second side 110 (e.g., a bottom side when the ostomy pouch 102 is oriented in its intended manner on the body of the patient), and a plurality of adjacent sides 112 interconnecting the first and second sides 108, 110, respectively (e.g., a remaining portion of the ostomy pouch 102 extending between the first side 108 and the second side 110). The first side 108 is configured to be attached to the patient via a stoma base plate 115.

The stoma base plate 115 of this embodiment may comprise either a one-piece system or a two-piece system and, in either case, will include a proximal opening 116 extending therethrough. The second side 110 includes a distal opening 118 (e.g., an outlet) through which a distal end 122 of the ostomy bag 104 extends therepast to couple to the cap 106. In a further embodiment, instead of the second side 110 including the distal opening 118, the ostomy pouch 102 may include a longitudinal slit that extends from the first side 108 to the second side 110 along one of the adjacent sides 112. The longitudinal slit may be opened and closed using a closing mechanism (e.g., zipper, Velcro, latch, etc.).

The ostomy pouch 102 is configured to house the ostomy bag 104 therein. In the exemplary embodiment, the ostomy bag 104 extends from a proximal end 120 to the distal end 122. The proximal end 120 of the ostomy bag 104 couples to the stoma via the stoma base plate 115. The effluent may then enter from the stoma into the ostomy bag 104 through the proximal opening 116 of the stoma base plate 115 and the proximal end 120 of the ostomy bag 104.

As shown in FIG. 2, the cap 106 is configured to couple to the ostomy pouch 102 with a portion of the ostomy bag 104 gripped therebetween. Specifically, the cap 106 is configured to couple over the distal opening 118 of the ostomy pouch 102 such that a protruding portion 122A of the distal end 122 of the ostomy bag 104 extends between the distal opening 118 and the cap 106. The protruding portion 122A of the distal end 122 of the ostomy bag 104 extends through the distal opening 118 of the ostomy pouch 102 and overlaps a portion of the second side 110 of the ostomy pouch 102 surrounding the distal opening 118.

In other words, the protruding portion 122A of the distal end 122 of the ostomy bag 104 lies in between the cap 106 and the ostomy pouch 102, surrounding the distal opening 118. Accordingly, although the cap 106 couples to the ostomy pouch 102, the cap 106 does so without directly contacting the ostomy pouch 102. As would be understood by those skilled in the art, the cap 106 may be at least partially transparent to form an inspection window for the patient to see the effluent in the pouch device 100.

As shown in FIG. 2, at least one of the cap 106 and the distal opening 118 may include a coupling mechanism 124 configured to couple the cap 106 over the distal opening 118. The coupling mechanism 124 forms a seal between the cap 106 and the ostomy pouch 102 to prevent effluent from leaking out of the ostomy bag 104. In an exemplary embodiment, the coupling mechanism 124 includes threading along an exterior surface of the second side 110 of the ostomy pouch 102 surrounding the distal opening 118. The cap 106 of such an embodiment includes corresponding threading along an interior portion thereof so that the cap 106 can be screwed over the protruding portion 122A of the distal end 122 of the ostomy bag 104 to secure the cap 106 relative to the ostomy pouch 102 and the ostomy bag 104.

The patient secures the cap 106 to the ostomy pouch 102 by rotating the cap 106 about its longitudinal axis (e.g., clockwise). In a further embodiment, the second side 110 of the ostomy pouch 102 may include, instead of the distal opening 118, a coupling mechanism 124. Further, at least one of the interior surface of the cap 106 and an exterior surface of the protruding portion 122A of the distal end 122 may include an adhesive to permanently attach the cap 106 to the ostomy bag 104. In a further embodiment, the interior surface of the cap 106 includes a heating mechanism or another mechanism to permanently attach the cap 106 to the ostomy bag 104 e.g., by melting and welding these elements together.

As shown in FIG. 25, the ostomy bag 104 of this embodiment is sized, shaped and configured to be separated into a plurality of sections 126 where each of the sections 126 is sized, for example, similarly to existing ostomy bags (e.g., ostomy bags that are completely removed when filled with effluent). In the present embodiment, the sections 126 of the ostomy bag 104 may be substantially similar in size with a distal-most section 126A (i.e., a first one of the sections 126 that is presently mounted over the cap 106).

The distal-most section 126A is configured to be filled and removed from a next adjacent section 126B (i.e., a second one of the sections 126) so that the adjacent section 126B can be positioned as described above to receive effluent from the stoma. The process is repeated for each of the sections 126 (e.g., the sections 126A, 126B, 126C, 126D, and 126E) until the section 126E (i.e., a fifth and last one the sections 126) has been filled. Once the section 126E is removed by the patient, a new multi-section ostomy bag is positioned with a distal-most section thereof mounted under the cap 106 as described above.

More specifically, the ostomy bag 104 includes the distal-most section 126A, which is the first one of the sections 126 to receive effluent from the stoma, and a plurality of remaining sections 125 (e.g., unused, empty, not distal-most sections) that are proximal of the distal-most section 126A. For example, when the distal-most section 126A is mounted under the cap 106 to receive effluent, the remaining sections 125 includes the sections 126B-126E. Each of the remaining sections 125, when the sections 126 distal thereof are filled with effluent, is configured to be positioned as described above to receive effluent from the stoma. For example, the section 126D, which is a third one of the remaining sections 125 and a fourth one of the sections 126, is mounted under the cap 106 to become a distal-most section after the first three sections 126, the sections 126A-126C, have been used and removed.

Each of the sections 126 is configured to be moved from a compacted (i.e., folded) configuration in which the sections 126 not currently in use (e.g., the remaining sections 125) are maintained, to an expanded (i.e., unfolded) configuration in which the section 126 becomes a distal-most section (e.g., the distal-most section 126A). That is, when one of the sections 126 immediately distal to another one of the sections 126 is removed, the another one of the sections 126 is drawn out and unfolded into the expanded configuration to become a new distal-most one of the sections 126.

For example, when the section 126C, which is immediately distal of the section 126D, is removed, the section 126D becomes a new distal-most section because the section 126E is proximal of the section 126D. When one of the sections 126 is in the expanded configuration, the one of the sections 126 is opened to receive effluent from the stoma through a proximal end 127 thereof. For example, as shown in FIG. 4, in the expanded configuration, a proximal end 127A of the distal-most section 126A is open to receive effluent from the stoma. In the compact configuration, the remaining sections 125 are not yet configured or positioned to receive effluent from the stoma. Thus, the remaining sections 125 are compacted to minimize the space required to house the remaining sections 125 in the ostomy pouch 102.

In an exemplary compacted configuration, a proximal end of a first one of the remaining sections 125 is adjacent to a distal end thereof and thus, minimizing the volume occupied by the first one of the remaining sections 125. This may be true for all of the remaining sections 125. When one of the remaining sections 125 is needed to replace a filled one of the sections 126, the one of the remaining sections 125 is pulled by the patient to unfold into the expanded configuration and this section is then positioned to receive effluent from the stoma in the manner described above.

The pouch device 100 may be used in the following manner. When the distal-most section 126A is filled with effluent 5, the patient may apply a force (e.g., a pulling force) on the cap 106 to separate the cap 106 from the distal opening 118 of the ostomy pouch 102. Once the cap 106 has been separated from the ostomy pouch 102, the patient pulls the filled distal-most section 126A past the distal opening 118 and detaches (e.g., cuts, tears) the filled distal-most section 126A from a remaining portion 105 of the ostomy bag 104, the remaining portion 105 including the remaining sections 125.

The patient then separates the filled distal-most section 126A from the remaining sections 125 (e.g., by cutting the ostomy bag 104 along the proximal end 127A of the filled distal-most section 126A). The ostomy bag 104 of an exemplary embodiment includes markings (e.g., a line A as shown FIG. 5) to guide the patient where to cut and detach the filled distal-most section 126A from the remaining sections 125. The proximal end 127A (i.e., proximal opening) of the filled distal-most section 126A may then be closed in any known manner (e.g., sealed, tied, clamped, etc.) and the filled distal-most section 126A may then be discarded in the same manner as with known ostomy bags. In an exemplary embodiment, the proximal end 127A of the filled distal-most section 126A is configured to be closed (e.g., sealed, tied, clamped, etc.) before the filled distal-most section 126A is cut by the patient and detached from the remaining sections 125.

Once the distal-most section 126A is detached from the ostomy bag 104, a next one of the sections 126 of the ostomy bag 104 (i.e., the first one of the remaining sections 125 immediately proximal to the distal-most section 126A) becomes the new distal-most section 126B. As one with ordinary skill in the art will understand, the new distal-most section 126B is the second one of the sections 126 and the first one of the remaining sections 125 but this same relationship will apply between each of the sections 126 as the section immediately distal thereto is filled and removed. A distal end 128B (i.e., distal opening) of the new distal-most section 126B is then be pulled out through the distal opening 118 of the ostomy pouch 102 and a new cap 106A is coupled to the distal opening 118 over the distal end of the new distal-most section 126B.

The new cap 106A is coupled to the distal opening 118, for example, in a manner substantially similar to that described for the coupling of the cap 106 to the distal opening 118. In the exemplary embodiment, the ostomy bag 104 includes five sections 126 (i.e., the sections 126A-126E) permitting the patient to use the ostomy bag 104 five times before having to load a new multi-section ostomy bag 104 at which time the patient may also replace the pouch device 100 if desired. Therefore, the embodiments described herein permit a patient to reduce the frequency with which the ostomy bag 104 (and, perhaps the pouch device 100) are replaced to, for example, once or twice a day as opposed to the 4-10 changes a day currently required with single-use ostomy bags. As would be understood by those skilled in the art, further exemplary embodiments may include multi-section ostomy bags 104 including more than or fewer than five sections 126 as desired. For example, a number of sections for an ostomy bag 104 may be increased until a space required to house the ostomy bag 104 exceeds a volume deemed acceptable by a patient.

The ostomy bag 104 may be formed of a pliable material (e.g., plastic or polymer) that permits the ostomy bag 104 to expand in volume when the ostomy bag 104 fills with the waste. The ostomy pouch 102 may similarly be formed of a pliable material (e.g., plastic or polymer). The pliable material of the ostomy pouch 102 may be different from the pliable material of the ostomy bag 104 so that the ostomy pouch 102 may provide more support for storing a greater length of the ostomy bag 104 or a greater number of the ostomy bags 104. For example, the pliable material of the ostomy pouch 102 may be more rigid and denser than the pliable material of the ostomy bag 104.

FIGS. 7-12 show an ostomy pouch device 200 according to a second exemplary embodiment of the present disclosure. The pouch device 200 is substantially similar to the pouch device 100, except as noted below. Similarly to the pouch device 100, the pouch device 200 includes an ostomy pouch 202 housing a multi-section ostomy bag 204 inside. Rather than a cap couplable to a distal end of an ostomy bag, the pouch device 200 includes an iris diaphragm 206 housed therein configured to seal of the ostomy bag 204 as will be described in greater detail below.

The ostomy pouch 202 is substantially similar to the ostomy pouch 102 of the pouch device 100 and extends from a first side 208 to a second side 210 and includes a plurality of adjacent sides 212 extending therebetween. The first side 208 is configured to be attached to the stoma and the patient via, e.g., a stoma base plate, and the second side 210 includes an outlet configured to be opened and closed by the patient via a closing mechanism (e.g., zipper, Velcro, latch, etc.) to access the ostomy bag 204 and the iris diaphragm 206 inside the ostomy pouch 202.

In a further embodiment, instead of the second side 210 configured to be open and closed, the ostomy pouch 202 may include a longitudinal slit that extends from the first side 208 to the second side 210 along one of the adjacent sides 212. The longitudinal slit of this embodiment is configured to be opened and closed using a closing mechanism (e.g., zipper, Velcro, latch, etc.). Further, a portion of each or some of the adjacent sides 212 may be at least partially transparent to form an inspection window for the patient to see the effluent in the pouch device 200.

The ostomy bag 204 is substantially similar to the ostomy bag 104 and is housed in the ostomy pouch 202 with the ostomy bag 204 extending from a proximal end 220 to the distal end 222. The proximal end 220 of the ostomy bag 204 is configured to be attached to the stoma via the stoma base plate. The ostomy bag 204 is separatable into a plurality of sections 226 in a manner similar to that described above in regard to the ostomy bag 104 with each of the sections 226 being substantially similar in size as described above. Each of the sections 226 is configured to be moved from a compacted (i.e., folded) configuration, when the section 226 is not in use, to an expanded (i.e., unfolded) configuration, when the section 226 is in use.

The ostomy bag 204 includes a distal-most section 226A, which is configured to be positioned so that it is the first one of the sections 226 to receive effluent from the stoma. A plurality of remaining sections 225 (e.g., unused, empty, not distal-most sections) extend proximally from the distal-most section 226A. Each of the remaining sections 225 is configured to be positioned to receive effluent from the stoma after the distal-most section 226A has been filled and removed, as will be described in greater detail below. Therefore, each of the sections 226 is configured to be filled, one at a time, with effluent that exits the patient through the stoma and passes through the proximal end 220 of the ostomy bag 204 and the iris diaphragm 206.

The iris diaphragm 206 is housed within the ostomy pouch 202 and may be configured to be attached to an interior surface of the ostomy pouch 202 or the stoma base plate. The iris diaphragm 206 includes a wall 230 and a plurality of blades 232 forming an aperture 234 that opens and closes as an iris in the same manner as the iris of a camera as would be understood by those skilled in the art. As would be understood by those skilled in the art, the iris diaphragm 206 is configured to be actuated between an open configuration and a closed configuration by rotating the wall 230 in a first direction (e.g., clockwise) relative to the ostomy bag 204. The iris diaphragm 206 may then be actuated from the closed configuration to the open configuration by rotating the wall 230 in a second direction (e.g., counter-clockwise) relative to the ostomy bag 204. In the open configuration, the blades 232 retract radially outward away from a longitudinal axis of the iris diaphragm 206 toward an interior portion of the wall 230.

The wall 230 may include a plurality of gripping features (e.g., textured features, raised bumps, raised ridges, knurled texture, etc.) for the patient to grip when twisting the wall 230 to actuate the iris diaphragm 206 between the open and closed configurations. In a further exemplary embodiment, the ostomy pouch 202 may have substantially similar gripping features extending about a portion of an outer surface thereof for the patient to use to actuate the iris diaphragm 206 between the open and closed configurations. The ostomy pouch 202 may further include an actuating mechanism (e.g., a button, a wheel, a lever, etc.) that actuates the iris diaphragm 206 between the open and closed configurations without needing the patient to mechanically twist the wall 230.

As shown in FIG. 10, the aperture 234 is at its maximum diameter (e.g., approximately 6 cm in diameter) when the iris diaphragm 206 is in the open configuration. When the iris diaphragm 206 is in the open configuration, the ostomy bag 204 is open to provide a clear path from the stoma into the ostomy bag 204 without the blades 232 or the iris diaphragm 206 interfering with the flow into the ostomy bag 204. In the closed configuration, the blades 232 extend toward the longitudinal axis of the iris diaphragm 206 from the interior portion of the wall 230 to close the opening in the ostomy bag 204 when, as shown in FIG. 11, the aperture 234 is at its minimum diameter (e.g., approximately 3 cm in diameter) and the iris diaphragm 206 is in the closed configuration. The aperture 234 may be sized and shaped based on a size of the stoma.

As shown in FIG. 12, when the iris diaphragm 206 is in the closed configuration, a portion of the ostomy bag 204 may be tightened by the blades 232 to separate a filled portion of the ostomy bag 204 from a remaining portion 205 of the ostomy bag 204. In this embodiment, the iris diaphragm 206 is coupled to the pouch device 200 (at any location within or external to the pouch device 200 selected as a location at which a proximal end 227A of the distal-most section 226A will be located when it is separated from the remaining portion 105 of the ostomy bag 204). Thus, when the distal-most section 226A of the ostomy bag 204 is ready to be changed, the patient pulls the ostomy bag 204 through the aperture 234 until the proximal end 227A of the distal-most section 226A is distal of the aperture 234. The patient then moves the iris diaphragm 206 to the closed configuration in which the aperture 234 is cinched down to grip the proximal end 227A of the distal-most section 226A.

For example, the patient closes the aperture 234 by rotating the wall 230 until the aperture 234 is completely closed around the proximal end 227A of the distal-most section 226A. The patient may then seal the proximal end 227A of the distal-most section 226A by any known means. For example, the patient may apply a mechanical fastener around the proximal end 227A of the distal-most section 226A (e.g., an elastic band, a zip tie, a heat seal, etc.) to ensure that the proximal end 227A of the distal-most section 226A seals the effluent therein. The distal-most section 226A may then be separated from the remaining portion 205 of the ostomy bag 204 (e.g., by cutting the distal-most section 226A away from the remaining sections 225) and an open distal end of the remaining portion 205 is sealed (e.g., using the same or a similar mechanism as was used to seal the proximal end 227A of the previous distal-most section 226A).

Similar to the pouch device 100, the pouch device 200 is configured to be used numerous times. That is, the ostomy pouch device may be used for as long as desired and/or may be changed, for example, each time a multi-section ostomy bag 204 that had been loaded therein has been completely used (i.e., when all of the sections 226 have been used and removed from the pouch device 200). Thus, a new ostomy pouch device 200 including a new multi-section ostomy bag 204 may be used.

Alternatively, a new multi-section ostomy bag 204 may be loaded into the pouch device 200 and this pouch device 200 may be reused as often as desired. As mentioned above, the ostomy bag 204 of this embodiment includes the plurality of sections 226 with the distal-most section 226A thereof separated from the remaining sections 225 by the iris diaphragm 206. To use the pouch device 200, the patient moves the iris diaphragm 206 into the open configuration and pulls the distal end 222 of the ostomy bag 204 through the aperture 234 of the iris diaphragm 206 until the distal end 222 is adjacent to the second side 210 of the ostomy pouch 202. Once the second side 210, or the longitudinal slit, is closed via the closing mechanism, the distal-most section 226A of the ostomy bag 204 rests in the ostomy pouch 202 ready to receive the effluent from the patient.

The pouch device 200 may be used in the following manner. Once the distal-most section 226A of the sections 226 is filled with effluent 15, the patient twists the wall 230 in the first direction to move the iris diaphragm 206 from the open configuration to the closed configuration. When the iris diaphragm 206 is rotated to the closed configuration, the blades 232 extending from the wall 230 move radially inward and apply a force on the proximal end 227A of the distal-most section 226A to tighten the proximal end 227A as the diameter of the aperture 234 decreases. When the proximal end 227A (i.e., proximal opening) has been tightened as desired (i.e., the proximal end 227A has been sealed), the patient opens the second side 210 of the ostomy pouch 202 to seal off the distal-most section 226A by permanently closing the proximal end 227A via a closing device 236. The closing device 236 may be any closing device known to one with ordinary skill in the art (e.g., tie, clip, clamp, heating device) that would seal the proximal end 227A to prevent the effluent 15 from escaping the distal-most section 226A.

Once the distal-most section 226A has been sealed off, the patient detaches the distal-most section 226A from the remaining sections 225 by cutting the ostomy bag 204 proximally of the proximal end 227A of the distal-most section 226A. The ostomy bag 204 of this embodiment includes optional markings (e.g., a line B) to guide the patient where to cut and detach the distal-most section 226A from the remaining sections 225. After the distal-most section 226A has been separated from the remaining sections 225 and discarded, the next most distal section of the sections 226 becomes the new distal-most section 226B. As one with ordinary skill in the art will understand, the new distal-most section 226B is a second one of the sections 226 and a first one of the remaining sections 225. This process is repeated until all of the sections 226 have been used and discarded.

The patient then rotates the wall 230 in the second direction to move the iris diaphragm 206 into the open configuration and pulls a distal end 228B of the new distal-most section 226B through the aperture 234 until the distal end 228B (i.e., distal opening) is adjacent to the second side 210 of the ostomy pouch 202. The patient may then seal the distal end 228B in a manner substantially similar to that described in regard to the sealing of the proximal end 227A of the distal-most section 226A to prevent further effluent from leaking out through the distal end 228B. The second side 210 may then be closed and the new distal-most section 226B is opened into the expanded configuration to receive effluent from the stoma.

In an exemplary embodiment, as shown in FIG. 11, the iris diaphragm 206 includes an integrated bag sealer 238. The bag sealer 238 is configured to seal proximal ends (e.g., the proximal end 227A) and distal ends (e.g., the distal end 228B) of the sections 226 so that effluent does not leak through the proximal and distal ends of the sections 226. The bag sealer 238 may include a heating element 240 formed, e.g., by or on the blades 232. The heating element 240 of this embodiment is activated by the patient to seal the proximal and distal ends of the sections 226 using heat. The heating element 240 (e.g., a wire) of this embodiment reaches a temperature high enough to at least partially melt the material of the ostomy bag 204 but without generating an amount of heat sufficient to damage the blades 232 or any other portion of the iris diaphragm 206.

The heating element 240 of an exemplary embodiment is powered by a power source (not shown) such as a battery coupled to the wall 230 of the iris diaphragm 206 or hidden within the wall 230. In a further exemplary embodiment, the bag sealer 238 seals the proximal and distal ends of the sections 226 using a closing device similar to the closing device 236. In a further exemplary embodiment, the iris diaphragm 206 further includes a bag cutter configured to be operated by the patient to cut and detach the sections 226 from each other. The bag cutter may be powered, for example, manually or via the power source powering the bag sealer 238 or via a further power source coupled to or hidden within the iris diaphragm 206 or at any other desired location.

In the exemplary embodiment, the ostomy bag 204, similar to the ostomy bag 104, includes five sections 226, such that the patient may use the ostomy bag 204 five times before having to replace the ostomy bag 204 or before having to replace the pouch device 200. As described above, this may permit the patient to replace the ostomy bag 204 and/or the pouch device 200 as little as once or twice a day instead of the 4-10 changes per day currently required with single-use ostomy bags. In a further exemplary embodiment, the ostomy bag 204 may include more than or less than five sections 226.

FIGS. 13-24 show an ostomy pouch device 300 according to a third exemplary embodiment. The ostomy pouch device 300 is substantially similar to the pouch devices 100, 200, except as noted below. Similarly to the pouch devices 100, 200, the ostomy pouch device 300 includes an ostomy pouch 302 housing an ostomy bag 304 inside. Rather than a cap or an iris diaphragm, the ostomy pouch device 300 includes a plurality of flanges 306 configured to grasp the ostomy bag 304 as will be described in greater detail below.

The ostomy pouch 302 is substantially similar to the ostomy pouches 102, 202 of the pouch devices 100, 200, respectively. The ostomy pouch 302 extends from a first side 308 to a second side 310 and includes a plurality of adjacent sides 312 therebetween. The first side 308 is configured to be attached to the stoma 20 and the patient via a stoma base plate 315 as described above. The ostomy pouch 302 of this exemplary embodiment further includes a longitudinal slit 303 (e.g., an outlet) extending from the first side 308 to the second side 310 along one of the adjacent sides 312. The longitudinal slit 303 is configured to be opened and closed using a closing mechanism (e.g., zipper, Velcro, latch, etc.) to permit the patient to access the ostomy bag 304 and the flanges 306 therein. A portion of one or more of the adjacent sides 312 may be at least partially transparent to form an inspection window for the patient to see the effluent in the ostomy pouch device 300.

The ostomy bag 304 is substantially similar to the ostomy bags 104, 204. The ostomy bag 304 is housed in the ostomy pouch 302 and extends from a proximal end 320 to the distal end 322. The proximal end 320 of the ostomy bag 304 is configured to be attached to the stoma 20 via the stoma base plate 315. The ostomy bag 304 may further include a first closing device 336 on the distal end 322 of the ostomy bag 304. The first closing device 336 may be any closing device known to one with ordinary skill in the art (e.g., tie, clip, clamp, heating device) that would seal the distal end 322 of the ostomy bag 304 and prevent effluent from escaping the ostomy bag 304.

The ostomy bag 304 is separatable into a plurality of sections 326, each of the sections 326 being substantially similar in size. The sections 326 are configured to be moved from a compacted (i.e., folded) configuration, when the sections 326 are not in use, to an expanded (i.e., unfolded) configuration, when the sections 326 are in use. The ostomy bag 304 includes a distal-most section 326A, which is the first one of the sections 326 to receive effluent from the stoma, and a plurality of remaining sections 325 (e.g., unused, empty, not distal-most sections) that are proximal of the distal-most section 326A.

The remaining sections 325 are configured to be folded up in the compacted configuration and held in place against the stoma base plate 315 by the flanges 306 extending therefrom. The remaining section 325 may be folded in, for example, an accordion-like style, as shown in FIG. 15 although, as would be understood by those skilled in the art, any other arrangement of any of the described ostomy bags may be employed so long as it permits the ostomy bag to be serially deployed in sections (i.e., a new section may be positioned to receive effluent after a more distal section has been filled and removed) and so that the volume required to house the multi-section ostomy bag is acceptable to the patient. The remaining sections 325 are configured to receive effluent from the stoma after the distal-most section 326A is filled, as will be described in greater detail below. Therefore, each of the sections 326 is configured to be filled, one at a time, with effluent that exits the patient through the stoma and passes through the proximal end 320 of the ostomy bag 304.

The flanges 306 extend outward from the stoma base plate 315. Each of the flanges 306 includes a curved portion 330 and a straight portion 332. The curved portion 330 extends longitudinally outward from the stoma base plate 315. The straight portion 332 extends radially inward from the curved portion 330 toward a longitudinal axis of the stoma base plate 315. Each of the flanges 306 is configured to move between a tightened configuration and a loosened configuration. In the tightened configuration, the flange 306 is bent radially inward to tightly secure the remaining sections 325 against the stoma base plate 315.

Specifically, in the tightened configuration, an angle α of the curved portion 330 is decreased so that the remaining sections 325 are fixed between the straight portion 332 and the stoma base plate 315. In the loosened configuration, the flange 306 is bent radially outward to release at least one section from the remaining sections 325 so that the at least one section is no longer held tightly against the stoma base plate 315. Specifically, in the loosened configuration, the angle α of the curved portion 330 is increased so that the at least one section, for example a next section 326B, is released from the flanges 306. The remaining sections 325 (i.e., those proximal of the next section 326B) may then be once again secured by moving the flanges 306 back into the tightened configuration.

The distal-most section 326A includes a proximal end 327A extending from the remaining sections 325 and a distal end 328A that includes, in one embodiment, a first closing device 336 thereon. The distal-most section 326A is the first one of the sections 326 to be filled with effluent from the patient. Once the distal-most section 326A is filled with effluent 25, as shown in FIG. 16, the patient may open the longitudinal slit 303 of the ostomy pouch 302 to access the flanges 306. The patient then moves the flanges 306 from the tightened configuration to the loosened configuration and pulls on the ostomy bag 304 until the next section 326B appears, as shown in FIG. 17. As one with ordinary skill in the art will understand, the next section 326B is a second one of the sections 326 and a first one of the remaining sections 325. The flanges 306 may then be moved from the loosened configuration to the tightened configuration to tightly secure the remaining sections 325 against the stoma base plate 315.

The proximal end 327A may then be twisted, as shown in FIG. 18, to isolate the distal-most section 326A from the next section 326B. The distal-most section 326A may then be sealed off from the next section 326B by applying a second closing device 338 on the proximal end 327A of the distal-most section 326A and a third closing device 340 on a distal end 328B of the next section 326B. The second and third closing devices 338, 340 may be substantially similar to the first closing device 336. Once the distal-most section 326A is sealed off, the patient may detach the distal-most section 326A from the next section 326B by cutting the ostomy bag 304 in between the distal end 328B of the next section 326B and the proximal end 327A of the distal-most section 326A, as shown in FIG. 19. Once the distal-most section 326A is detached and discarded, the next section 326B, as shown in FIG. 20, may be opened into the expanded configuration to receive further effluent from the patient.

As indicated above, the ostomy bag 304 may include markings (e.g., dashed lines) indicating desired locations at which the sections 326 may be separated from one another and to guide the patient where to cut the ostomy bag 304 to detach the sections 326 from one another. For example, a line C may be printed on the ostomy bag 304 to separate the distal-most section 326A from the next section 326B. The patient may orientate himself/herself to place the second closing device 338 distal of the line C and the third closing device 340 proximal of the line C. The patient may then cut the ostomy bag 304 along the line C to detach the distal-most section 326A from the next section 326B. Further, the markings may be used to guide the patient on a length of the ostomy bag 304 that should be pulled out from under the flanges 306 when the flanges 306 are in the loosened configuration, as in FIG. 17.

In a further embodiment shown in FIGS. 21-24, the ostomy bag 304 includes an irrigation hole 350 extending through a proximal portion of the distal-most section 326A, distal of the proximal end 327A. The irrigation hole 350 is configured to be penetrated by a blunt tip 352 of a syringe 354. The patient may insert the blunt tip 352 into a container holding a fluid (e.g., distilled water, cleaning solution, etc.) and pulls a plunger 356 of the syringe 354 to suction the fluid into a reservoir 358 of the syringe 354. In a further exemplary embodiment, the ostomy bag 304 may include a thin layer of material covering the irrigation hole 350, the thin layer configured to be punctured by the blunt tip 352, a sharp tip, or a semi-sharp tip of the syringe 354.

The patient then inserts the blunt tip 352 into the irrigation hole 350 and directs the blunt tip 352 toward the stoma 20, as shown in FIG. 21. The patient may then push down on the plunger 356 to compress the reservoir 358 until the fluid is expelled from the blunt tip 352 to irrigate the stoma 20 and the surrounding area. The fluid, and any stoma output, is flushed out by the fluid into the distal-most section 326A. The patient may irrigate the stoma 20 and the surrounding area until the distal-most section 326A is sufficiently full. The syringe 354 may be any syringe known to one with ordinary skill in the art that is capable of suctioning and ejecting fluid through a tip. Further, the tip may be blunt, sharp, or in between.

When the distal-most section 326A is full, the patient may detach and discard the distal-most section 326A in a substantially similar manner as above. Specifically, once the distal-most section 326A has become filled with effluent 35, as shown in FIG. 21, the patient opens the longitudinal slit 303 of the ostomy pouch 302 to access the flanges 306. The patient then moves the flanges 306 from the tightened configuration to the loosened configuration and pulls on the ostomy bag 304 until the next section 326B appears, as shown in FIG. 22. The flanges 306 may then be moved from the loosened configuration to the tightened configuration to tightly secure the remaining sections 325 against the stoma base plate 315. The patient then seals off the distal-most section 326A by applying the second closing device 338 to the proximal end 327A distal of the irrigation hole 350 and cuts the ostomy bag 304 along the line C to detach the distal-most section 326A from the next section 326B.

As shown in FIG. 23, after the distal-most section 326A has been detached and discarded, the distal end 328B of the next section 326B is left open so that the stoma 20 and the surrounding area may be dried by the patient. Once the stoma 20 and the surrounding area is dry, the patient may close the distal end 328B with the third closing device 340, as shown in FIG. 24, and the next section 326B is ready to receive effluent through the stoma 20. The longitudinal slit 303 of the ostomy pouch 302 is then closed so that the ostomy bag 304 to hold the ostomy bag 304 therein.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

1-14. (canceled)

15. An ostomy pouch device, comprising:
an ostomy pouch extending from a proximal end configured to attach to skin surrounding a stoma exit of a patient to a distal end, the ostomy pouch including an outlet accessible to the patient;
an ostomy bag housed within the ostomy pouch, the bag extending from a proximal end configured to interface with the stoma exit to a distal end, the bag including a plurality of sections separatable from one another; and
a securing mechanism configured to secure, relative to the ostomy pouch, remaining sections of the bag that are proximal of a distal-most section of the bag,
wherein the bag is configured so that, when the distal-most section is filled with waste from the stoma exit, the distal-most section may be detached from the remaining sections and a distal-most one of the remaining sections is configured to be secured by the securing mechanism relative to the ostomy pouch in a position to interface with the stoma exit to receive further waste from the stoma exit.

16. The ostomy pouch device of claim 15, wherein the securing mechanism includes a cap configured to couple the distal end of the bag to the distal end of the ostomy pouch.

17. The ostomy pouch device of claim 16, wherein the cap includes an adhesive to permanently attach the cap to the distal end of the bag.

18. The ostomy pouch device of claim 16, wherein at least one of the cap and the distal end of the ostomy pouch includes a coupling mechanism configured to couple the cap to the distal end of the bag.

19. The ostomy pouch device of claim 16, wherein the outlet is an opening in the distal end of the ostomy pouch.

20. The ostomy pouch device of claim 15, wherein the securing mechanism includes an iris diaphragm configured to be actuated between an open configuration, in which blades of the iris diaphragm are rescinded therein, and a closed configuration, in which the blades of the iris diaphragm are extended radially therefrom.

21. The ostomy pouch device of claim 20, wherein, when the iris diaphragm is in the closed configuration, the blades tighten a proximal end of the distal-most section to isolate the distal-most section from the remaining sections.

22. The ostomy pouch device of claim 20, wherein the iris diaphragm includes at one of a heating element and a cutting element so that, when the iris diaphragm is in the closed configuration, the blades tighten a proximal end of the distal-most section to at least one of seal the distal-most section from the remaining sections and detach the distal-most section from the remaining sections.

23. The ostomy pouch device of claim 15, wherein the securing mechanism includes a plurality of flanges configured to be actuated between a tightened configuration, in which flanges secure the remaining sections against a stoma base plate of the ostomy pouch, and a loosened configuration, in which the flanges release at least a portion of the remaining sections.

24. The ostomy pouch device of claim 15, further comprising a closing device configured to seal a proximal end of the distal-most section.

25. The ostomy pouch device of claim 24, further comprising a plurality of further closing devices each configured to seal at least one of a distal end of the distal-most section, a proximal end of each one of the remaining sections, and a distal end of each one of the remaining sections.

26. The ostomy pouch device of claim 15, further comprising an irrigation hole configured to be penetrated by an irrigation device to irrigate the stoma exit and a surrounding area of the stoma exit.

27. The ostomy pouch device of claim 26, wherein the irrigation hole is distal of a proximal end of the distal-most section.

28. An ostomy pouch device, comprising:
an ostomy pouch including a base plate configured to attach to skin surrounding a stoma exit, the ostomy pouch including an outlet;
an ostomy bag housed within the ostomy pouch and configured to interface with the stoma exit, the bag including a plurality of sections, each of the sections movable from a compacted configuration, in which the section is unable to receive waste therein, to an expanded configuration, in which the section is able to receive waste therein; and
a securing mechanism configured to secure at least one of a first one of the sections and a second one of the sections, relative to the ostomy pouch, the first one of the sections being distal of the second one of the sections,
wherein the bag is configured so that, when the first one of the sections is filled with waste from the stoma exit, the first one of the sections may be detached from the second one of the sections, and the second one of the sections is secured by the securing mechanism relative to the ostomy pouch and is moved from the compacted configuration to the expanded configuration to receive further waste from the stoma exit.

29. A method, comprising:
attaching to a stoma exit an ostomy pouch of an ostomy pouch device, the ostomy pouch device comprising an ostomy bag housed inside the ostomy pouch including a plurality of sections separatable from one another, each of the sections movable from a compacted configuration, in which the sections are unable to receive waste therein, to an expanded configuration, in which the sections are able to receive waste therein and a securing mechanism configured to secure, relative to the ostomy pouch, remaining sections of the bag that are proximal of a distal-most section of the bag;
opening an outlet of the ostomy pouch to access the bag and the securing mechanism;
actuating the securing mechanism to isolate the distal-most section;
sealing a proximal end of the distal-most section from the remaining sections;
detaching the distal-most section from the remaining sections;
securing, by the securing mechanism, relative to the ostomy pouch a first one of the remaining sections;
moving the first remaining section into the expanded configuration to receive the waste therein; and
sealing the outlet of the ostomy pouch.

30. The method of claim 29, wherein the step of sealing the proximal end of the distal-most section comprises attached a closing device onto the proximal end of the distal-most section.

31. The method of claim 29, further comprising:
attaching a closing device to a distal end of the first one of the remaining sections when the first one of the remaining sections is moved into the expanded configuration.

32. The method of claim 29, further comprising:
inserting an irrigation device into an irrigation hole in the bag to irrigate the stoma exit and a surrounding area of the stoma exit.

33. The method of claim 29, wherein the securing mechanism includes one of (a) a cap configured to couple a distal end of the bag to a distal end of the ostomy pouch, (b) an iris diaphragm configured to be actuated between an open configuration in which blades of the iris diaphragm are rescinded therein and a closed configuration in which the blades of the iris diaphragm are extended radially therefrom to tighten the proximal end of the distal-most section and isolate the distal-most section from the remaining sections, and (c) a plurality of flanges configured to be actuated between a tightened configuration in which flanges secure the remaining sections against a stoma base plate of the ostomy pouch, and a loosened configuration in which the flanges release at least a portion of the remaining sections.

34. The method of claim 29, wherein the distal-most section is detached from the remaining sections by cutting or tearing along the proximal end of the distal-most section.
